(19) United States
(12) Patent Application Publication
(10) Pub. No.: US 2007/0023049 A1
(43) Pub. Date: Feb. 1, 2007

(54) Anatomically hypopharyngeal mask

(76) Inventor: Shih-Chang Chen, Yuan Lin Town (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/188,755

(22) Filed: Jul. 26, 2005

(51) Int. Cl. A61M 16/00

(52) U.S. Cl. 128/207.15

(57) ABSTRACT

An anatomically hypopharyngeal mask, and particularly an anatomically hypopharyngeal mask with a shape closer to the human hypopharynx, may be placed into a patient's hypopharynx so that the gas for medical treatment may be smoothly conducted into the patient's trachea. The mask has an air intake tube and a mask. One side of the mask has two preformed first clamping portions protruding from two sides below an edge of the concavity of the mask. The anatomically hypopharyngeal mask may be precisely positioned in a predetermined site of the patient's hypopharynx and then the mouth of the mask is easily aimed at the patient's trachea. Placement time for the mask is thus shortened. Because the anatomically hypopharyngeal mask is closer in shape to that of the human hypopharynx, the air tight capability thereof is better.

### BACKGROUND OF THE INVENTION

1. Field of the Invention

This invention relates to an anatomically hypopharyngeal mask and particularly to an anatomically hypopharyngeal mask to be placed into a patient's hypopharynx so that gas for medical treatment may be smoothly conducted into the patient's trachea.

2. Description of Related Art

A particular medical treatment, for example, administration of general, gas-based anesthesia during a surgical operation or cardiopulmonary resuscitation (CPR), a patient's respiratory tract must be smooth, and a conventional endotracheal tube is widely used. However, a plug-in laryngeal mask has gradually come into use in recent years and, as shown in FIG. 1, the conventional laryngeal mask 9 is provided with an air intake tube 91. One end of the air intake tube 91 is connected with a mask 92. A bottom of the mask 92 is formed with a concavity 93, in which a plurality of openings 94 connects to the air intake tube 91. The circumference of mask 92 is provided with an approximately oval-shaped air inflated ring 95, of which a back end is connected through a gas tube 96 to an inflation bag 97. A valve 98 is located in a back end of the inflation bag 97.

Before intubation is performed, the gas in the air inflated ring 95 must be completely discharged and then the entire mask 92 is inserted into the patient's hypopharynx. A front end of the mask 92 is then placed against the upper opening of the esophagus so that the openings of the laryngeal mask are aimed at the patient's trachea. Then, an injector is used to allow air to be input into the air inflated ring 95 from the gas tube 96 through the valve 98 of the inflation bag 97 so that the air inflated ring 95 is inflated between the pharynx and the mask 92. No gap is thus formed between the patient's hypopharynx and the mask 92. The gas (anesthetic gas) for medical treatment can thus be smoothly delivered to the patient's trachea from the air intake tube 91 without leakage of the gas from gap. Also, according to a condition of inflation or deflation of the inflation bag 97, an inflation status of the inflated ring may be known.

However, when the conventional laryngeal mask mentioned above is used, the mask must be inserted into the patient's hypopharynx and the openings 94 are led to aim exactly at the patient's trachea so that the gas for medical treatment may be conducted into the patient's trachea. At the time of actual operation, however, the openings 94 of the mask 92 is difficult to adjust to aim at the patient's trachea due to a difficult visual check. The procedure is performed by medical personnel dependent on their sense of touch and on respective experience, and the procedure may be repeated many times for successful placement. This makes the patient uncomfortable and delays establishment of a clear airway.

Furthermore, the above-mentioned conventional laryngeal mask, due to a larger length and width thereof, is often difficult to place in the patient's hypopharynx and hindered an enlarged volume due to air inflation from pressurization in order to at least prop up to the pharynx. The mucous membrane of pressured portion is consequently hurt because a higher airtight capability is given, or air leakage occurring due to incomplete air inflation in order to protect the mucous membrane.

Refer to U.S. Pat. No. 6,705,318 disclosed a method for fabricating low cost laryngeal mask devices includes providing a mold, the mold including interior walls that define a hollow interior volume. The interior volume includes a first portion and a second portion. A liquid plastic material is introduced into the mold, and then the mold is moved so as to coat the mold's interior walls. The liquid plastic material and then allowed to cure. The cured plastic material is then removed from the mold and the cured plastic material includes a generally elliptically shaped plate and a cuff. The cuff is formed from plastic material that coated the portion of the interior walls that defined the first portion. The plate defines a laryngeal side, a pharyngeal side, and a central aperture. An interior perimeter of the cuff is attached to the laryngeal side of the plate proximal to a perimeter of the central aperture. An outer perimeter of the cuff is attached to the laryngeal side of the plate proximal to an outer perimeter of the plate.

Refer to U.S. Pat. No. 6,705,322 disclosed a laryngeal mask tube consists of a dual-airway tube, a mask with an inflatable bladder and an inflation indicator device. The dual-airway tube comprises a simplified primary tube and secondary tube integrally combined together. The primary tube communicates with the mask to guide gas into the body of a patient and the secondary tube communicates between with the bladder and the inflation indicator device to monitor and inflate the bladder. Moreover, two ribs are formed in the mask to prevent blockage of the primary tube and a tongue is formed inside the bladder to prevent the bladder from folding and preventing a seal around the larynx. Whereby, the laryngeal mask airway is convenient and efficient to use.

Refer to U.S. Pat. No. 5,743,258 provided a pharyngeal airway tube, without inserting any foreign matters in the trachea can be easily inserted through the oral cavity in the lower pharynx and is capable of hermetically sealing a transitional region from the lower pharynx to the esophagus and the periphery of the laryngeal opening to secure an airway for positive pressure artificial ventilation. The pharyngeal airway comprises an airway tube having a closed, round distal end and provided in its side wall with ventilating holes and a balloon attached to the edge of the ventilating part and around the ventilating part of the airway tube and capable of being inflated so as to surround a part of the airway tube near the distal end of the same. A small bore gas passage is formed in the wall of the airway tube so as to open into the balloon, and a small bore tube fitted with a connector is connected to the proximal end of the gas passage.

Refer to U.S. Pat. No. 4,509,514 provided an artificial airway device, for use in place of an endotracheal tube to facilitate lung ventilation in an unconscious patient, is in the form of a laryngeal mask comprising a tube opening into the interior of a mask portion whose periphery, which may be inflatable, is adapted to seal around the inlet to the larynx, thus securing the patient's airway, permitting spontaneous or controlled ventilation and preventing inhalation of extraneous matter.

### SUMMARY OF THE INVENTION

This invention is mainly to provide an anatomically hypopharyngeal mask which is easily and precisely oriented to a predetermined portion of a patient's hypopharynx so that the openings 94 of the mask is easily aimed at the patient's trachea. Thus, a clear airway can be more quickly established.

This invention provides an anatomically hypopharyngeal mask that more closely fits the hypopharyngeal structure, so the mask is smaller than a conventional laryngeal mask in length and width. The total volume and the cross-sectional area are smaller, and thus the mask is more easily placed into the patient's hypopharynx. It also does less harm the patient's hypopharyngeal mucosa because the volume thereof is not enlarged to at least prop up the hypopharynx to create an airtight seal.

In order to achieve the object mentioned above, this invention provides an anatomically hypopharyngeal mask comprising an air intake tube 91 and a mask connected to an end of the air intake tube 91. The mask and the air intake tube are formed communicating with each other. One side of the mask is formed with a concavity with at least a mouth connected to the air intake tube, while the other side of the mask is provided with two preformed first clamping portion protruding from two sides below the edge of mask.

In order to further know the features and technical means of this invention, a detailed description according to this invention accompanied by drawings is given; however, the accompanying drawings are provided for reference and illustration only and are not limitations on this invention.

### BRIEF DESCRIPTION OF THE DRAWINGS

The foregoing aspects and many of the attendant advantages of this invention will be more readily appreciated as the same becomes better understood by reference to the following detailed description, when taken in conjunction with the accompanying drawings, wherein:

FIG. 1 is a 3D view of a conventional laryngeal mask;

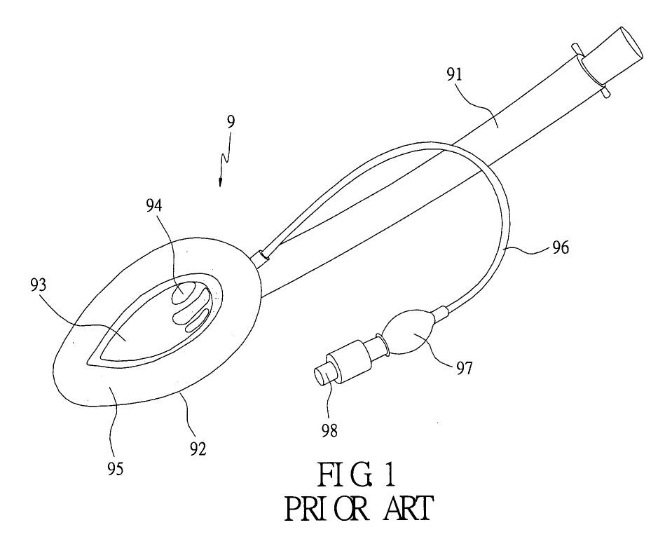

FIG. 2 is a 3D view of an anatomically hypopharyngeal mask according to a first embodiment of this invention;

FIG. 3 is a 3D view of the anatomically hypopharyngeal mask from another angle according to the first embodiment of this invention;

FIG. 4 is a side view of the anatomically hypopharyngeal mask according to the first embodiment of this invention;

FIG. 5 is a schematic view of the anatomically hypopharyngeal mask under use according to the first embodiment of this invention;

FIG. 6 is a 3D view of the anatomically hypopharyngeal mask according to a second embodiment of this invention;

FIG. 7 is a schematic view illustrating the anatomically hypopharyngeal mask under use according to the second embodiment of this invention; and

FIG. 8 is a 3D view of the anatomically hypopharyngeal mask according to a third embodiment of this invention.

### DETAILED DESCRIPTION OF PREFERRED EMBODIMENTS

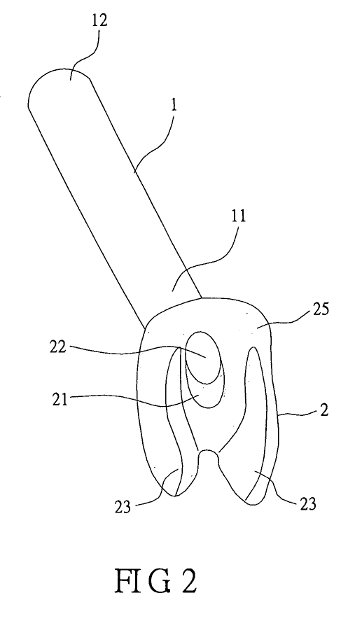

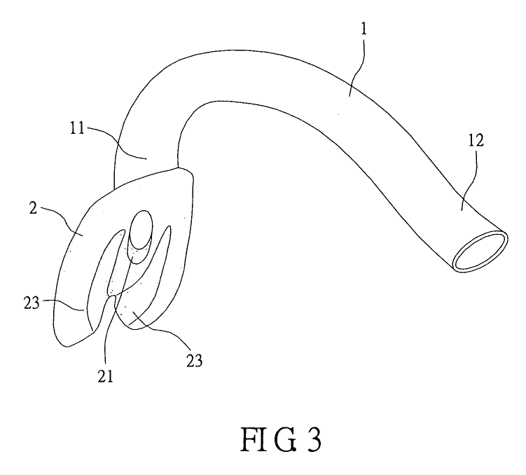

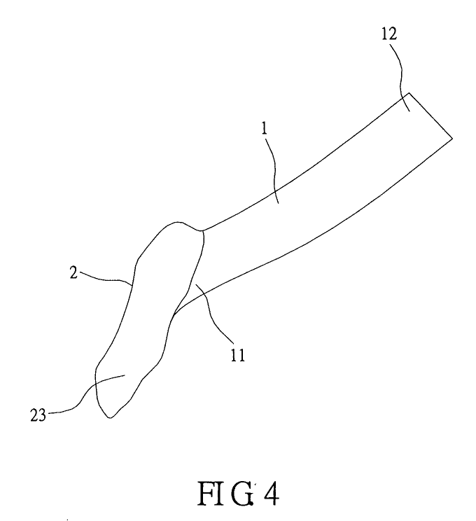

Referring now to FIGS. 2, 3 and 4, this invention provides an anatomically hypopharyngeal mask comprising an air intake tube 1 and a mask 2. The air intake tube 1 is flexible and properly bendable; two ends thereof are formed respectively into a first end 11 and a second end 12, of which the first end 11 is used to connect with the mask 2 and the second end 12 may be used to connect with a standard contact to output gas (anesthetic gas) for medical treatment.

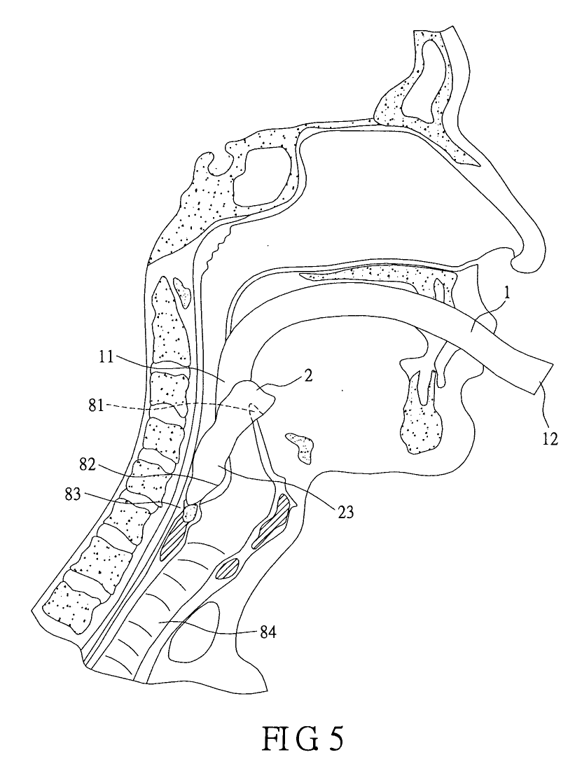

The mask 2 is made of silica gel as a soft material, the body 25 of mask is mostly of a rectangle of round obtuse angle, the mask 2 is connected to the first end 11 of the air intake tube 1, and the mask 2 and the air intake tube 1 communicate with each other. One side of the mask 2 is formed with a concavity 21 in which at least a mouth 22 connected to the air intake tube 1 is provided so that the gas for medical treatment may be delivered to the concavity 21 of the mask 2 from the air intake tube 1 and the mouth 22. One side of the mask 2 is also formed with two preformed first clamping portions 23 protruding from two sides below the edge of concavity 21 of the mask 2, and the preformed two clamping portions 23 protruding correspond to two concave portions (pyriform sinuses) 82 below the edge of the back side of the epiglottic cartilage of human body (FIG. 5). From the composition mentioned above, the anatomically hypopharyngeal mask according to this invention is made.

Referring now to FIG. 5, when intubation is performed, the mask of the anatomically hypopharyngeal mask according to this invention is fully placed into the hypopharynx and a front end of the mask 2 is located against the esophagus 83 so that the mouth 22 is aimed at the patient's trachea 84 to provide gas (anesthetic gas) for medical treatment to the patient's trachea from the air intake tube 1. The protruding mask 2 according to this invention is provided with the two preformed first clamping portions 23, so when the mask 2 of hypopharyngeal mask is placed into a determined portion of the patient's hypopharynx, the two preformed first clamping portions 23 correspond exactly to two concave portions (pyriform sinuses) 82 below the edge of the back side of the epiglottic cartilage 81 of human body. Orientation is thus achieved to position accurately the anatomically hypopharyngeal mask in a predetermined site of the patient's hypopharynx and the mouth 22 is lead to aim exactly at the patient's trachea so that the gas for medical treatment may be introduced into the patient's trachea. Through a design on the two preformed first clamping portions 23, the mouth 22 of mask 2 is made to aim easily and directly at the patient's trachea.

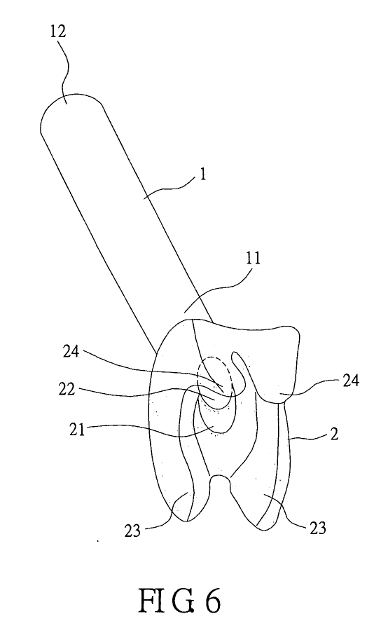

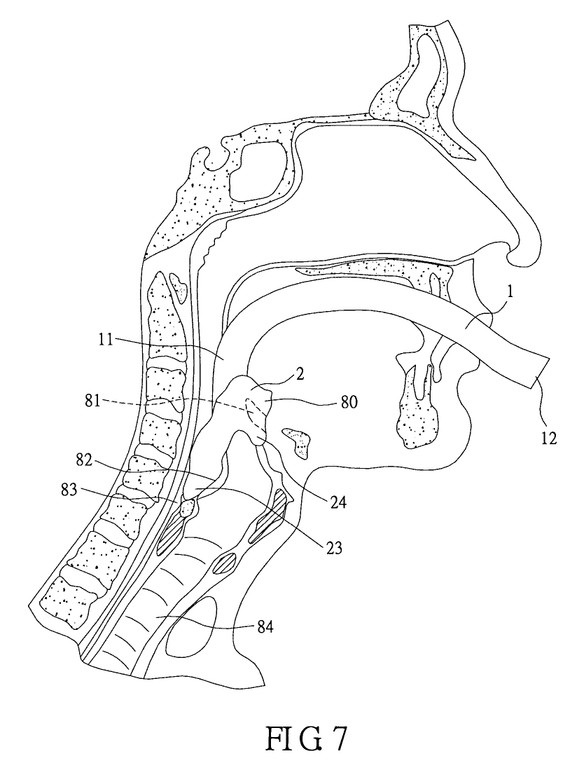

Further, referring now to FIGS. 6 and 7, in a second embodiment of this invention, one side of the mask 2 is also formed with two preformed second clamping portions 24 protruding from two sides above the edge of concavity 21 of the mask 2, and the preformed two clamping portions 24 protruding correspond to two concave portions (vallecula epiglottis) 80 above the edge of the front side of the epiglottic cartilage 81 of human body. When the mask 2 of the anatomically hypopharyngeal mask is placed into a determined portion of the patient's hypopharynx, the two preformed second clamping portions 24 are exactly placed across two concave portions (vallecula epiglottis) 80 above the edge of the front side of the epiglottic cartilage. A further orientation is thus also achieved to position accurately the anatomically hypopharyngeal mask in a predetermined site of the patient's hypopharynx and the mouth 22 is led to exactly aim at the patient's trachea.

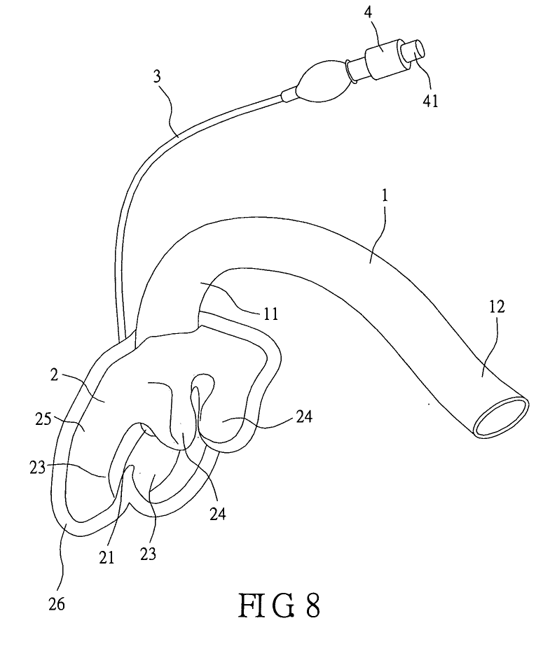

Next, referring now to FIG. 8, in a third embodiment of this invention, the circumference of mask 2 is further provided with an air inflation membrane 26 including a back end connected through a gas tube 3 to an inflation bag 4, a back end which has a valve 41. Before intubation is performed, the gas in the air inflation membrane 26 must be discharged and then the entire mask 2 is inserted into the patient's hypopharynx. Next, the front end of the mask 2 is placed against the esophagus so that the mouth aims at the patient's trachea. Then, an injector may be used to input air into the air inflation membrane 26 from the gas tube 3 through the valve 41 of the inflation bag 4 so that the air inflation membrane 26 is inflated to fill between the hypopharynx and the mask 2. No gap is thus formed between the patient's hypopharynx and the mask 2; the gas (anesthetic gas) for medical treatment is thus smoothly delivered to the patient's trachea from the air intake tube 1 without leakage of the gas from a gap. Simultaneously, according to inflation or deflation of the inflation bag 4, an air inflation status of the air inflation membrane 26 may be known.

The anatomically hypopharyngeal mask according to this invention provides at least the advantages set forth below:
1. The anatomically hypopharyngeal mask according to this invention is particularly designed with the two preformed clamping portions 23 to position accurately the anatomically hypopharyngeal mask in the predetermined site so that the mouth 22 of mask 2 is easily aimed at the patient's trachea. Easy placement of the anatomically hypopharyngeal mask prevents the patient from being uncomfortable and shortens time for establishment of a clear airway.
2. The anatomically hypopharyngeal mask according to this invention is much smaller than the conventional laryngeal mask in length and width, and even in volume and cross-sectional area, so as to be easily placed into the patient's hypopharynx.
3. The anatomically hypopharyngeal mask according to this invention is designed in accordance with ergonomics on a shape closer to the real shape of the hypopharynx than the conventional laryngeal mask.
4. The anatomically hypopharyngeal mask according to this invention matches the form of a normal human body dependent on a 3D geometrical construction instead of volume for a high airtight capability.
5. The anatomically hypopharyngeal mask according to this invention is comparatively less damaging to the patient's oral mucosa.
6. The anatomically hypopharyngeal mask according to this invention allows a nasogastric tube to be easily inserted directly from a reserved space of a backside.

In the description mentioned above, only the preferred embodiments according to this invention are provided without limit to claims of this invention; all those skilled in the art without exception should include the equivalent changes and modifications as falling within the true scope and spirit of the present invention.

What is claimed is:

1. An anatomically hypopharyngeal mask comprising:
an air intake tube; and
a mask connected to an end of the air intake tube, the mask and the air intake tube being formed communicating with each other, one side of the mask being formed with a concavity with at least a mouth connected to the air intake tube, and another side of the mask being provided with two preformed first clamping portions protruding from two sides below an edge of the concavity of the mask.

2. The anatomically hypopharyngeal mask according to claim 1, wherein one side of the mask is also formed with two preformed second clamping portions protruding from two sides above the edge of concavity of the mask.

3. The anatomically hypopharyngeal mask according to claim 1, wherein a circumference of the mask is provided with an air inflation membrane with a back end connected through a gas tube to an inflation bag with a back end having a valve.
